(19) United States
(12) Patent Application Publication
(10) Pub. No.: US 2008/0312524 A1
(43) Pub. Date: Dec. 18, 2008

(54) Medical Sensor Having Electrodes and a Motion Sensor

(75) Inventors: Thomas Solosko, Seattle, WA (US); Thomas Lyster, Bothell, WA (US); Steve Hugh, Bellevue, WA (US); Brett Cross, Andover, MA (US); Stacy Gehman, Seattle, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., Eindhoven (NL)

(21) Appl. No.: 12/095,792

(22) PCT Filed: Dec. 2, 2006
(86) PCT No.: PCT/IB2006/054563

§ 371 Date: Jun. 2, 2008

Related U.S. Application Data
(60) Provisional application No. 60/748,916, filed on Dec. 8, 2005.

(51) Int. Cl. A61B 5/04

(52) U.S. Cl. 600/393

(57) ABSTRACT

A medical sensor having at least one electrode configured to be placed on a patient for medical monitoring and a motion sensor integrated in the medical sensor with the electrode, the motion sensor configured to detect patient motion and provide electrical signals in response thereto.

The present invention relates generally to medical sensors, and more specifically, to medical sensors for sensing patient biological information and sensing patient motion.

For a number of years, cardiac patients have been evaluated with a cardiac monitoring/recording device known as a “Holter” electrocardiograph. A patient wears medical sensors, typically electrodes, that are connected to a portable recording device carried by the patient which records electrocardiograph (“ECG”) signals detected by the sensors. The patient ECG is recorded over a period of time, such as 24 hours, so that a record of heart activity over an extended time period can be obtained.

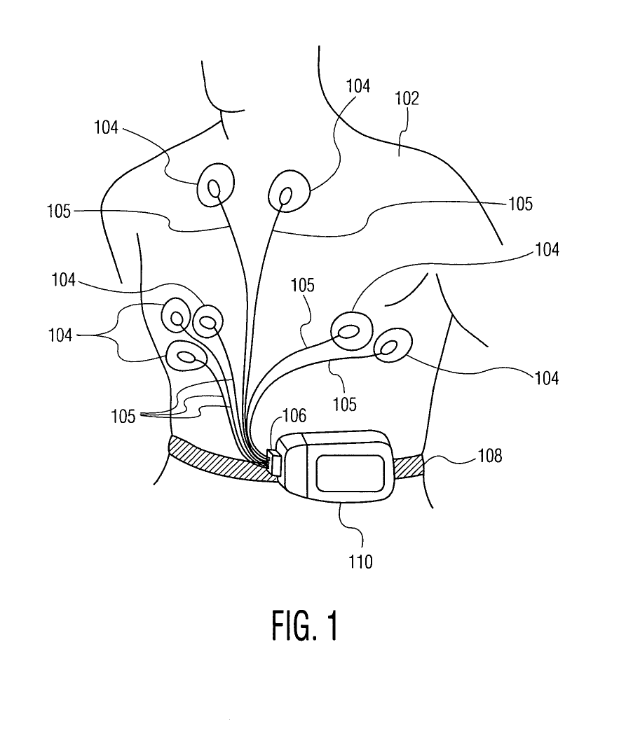

FIG. 1 illustrates a patient 102 wearing a Holter electrocardiograph. Medical sensors in the form of conventional electrodes 104 are attached to the patient 102 and are electrically coupled to a recorder 110 through wires 105 and connector 106. For clarity of illustration the number and placement of electrodes shown in FIG. 1 may differ from an actual patient configuration. The recorder 110 is typically worn by the patient 102 using a belt 108, or other means, such as being carried over the shoulder. The electrodes 104 detect electrical signals that are indicative of patient biological information and the recorder 110 records the electrical signals for later download and analysis. As shown in FIG. 1, conventional Holter electrocardiographs are bulky and conspicuous since the recorder 110 is typically too large to be worn comfortably under clothing. Moreover, the recorder 110 connects to the electrodes 104 through several wires 105, which can become tangled while the patient 102 is moving and can also increase discomfort when the wires 105 are worn under clothing.

In collecting and recording patient biological information, it can be helpful in interpreting the recorded information to also have information related to the motion of the patient. For example, the motion of a patient can be indicative of patient health, such as, patient motion suggesting that the patient is active and not in cardiac arrest, patient respiration, or patient heartbeats. Additionally, sensed motion, or lack thereof, can also serve as a quality indicator of cardiac signals, where motion can generate electrical signals that interfere with ECG signals. A motion sensor can be included in the recorder 110 to detect and record patient motion. However, due to the fact that the recorder 110 is relatively bulky, and is typically worn on the belt 108 or carried by a strap over the shoulder, the motion of the recorder 110 is not necessarily the motion of the patient, which may lead to recording inaccurate motion information.

One aspect of the invention provides a medical sensor having at least one electrode configured to be placed on a patient for medical monitoring and a motion sensor integrated in the medical sensor with the electrode. The motion sensor is configured to detect patient motion and provide electrical signals in response thereto.

Another aspect of the invention provides a medical sensor having a plurality of electrodes configured for placement on a patient and operable to electrically couple electrical signals to and from the patient. The medical sensor further includes an integral motion sensor configured to sense patient motion and provide signals in response thereto. Another aspect of the invention provides a method of forming a medical sensor including integrating a motion sensor and a plurality of electrodes in the medical sensor.

In the Drawings:

FIG. 1 is schematic representation of a conventional cardiac monitoring and recording system having conventionally configured electrodes.

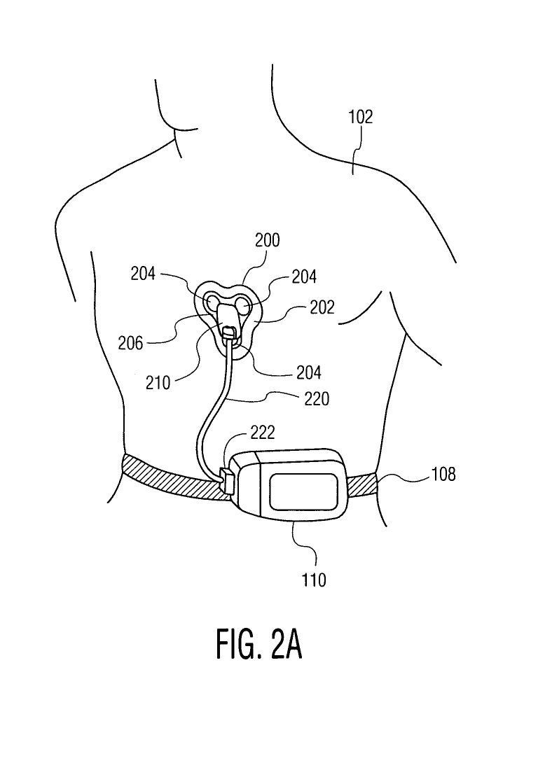

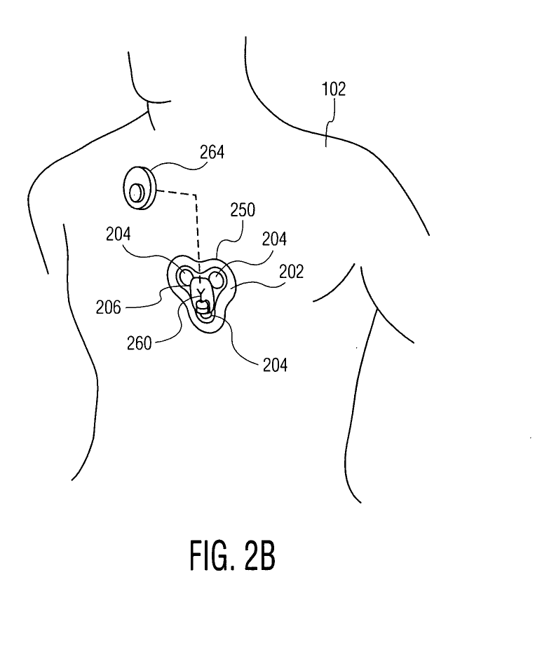

FIGS. 2A and 2B are schematic representations of a cardiac monitoring and recording system including a medical sensor according to embodiments of the present invention.

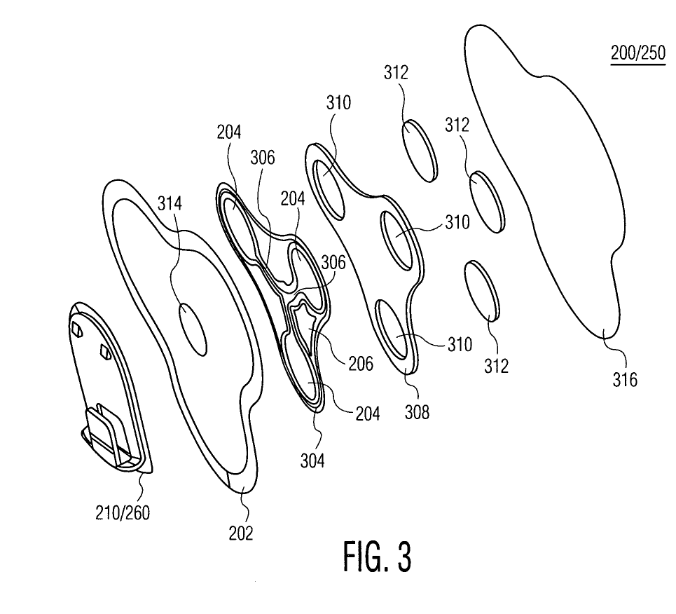

FIG. 3 is an exploded isometric diagram of the medical sensor of FIG. 2.

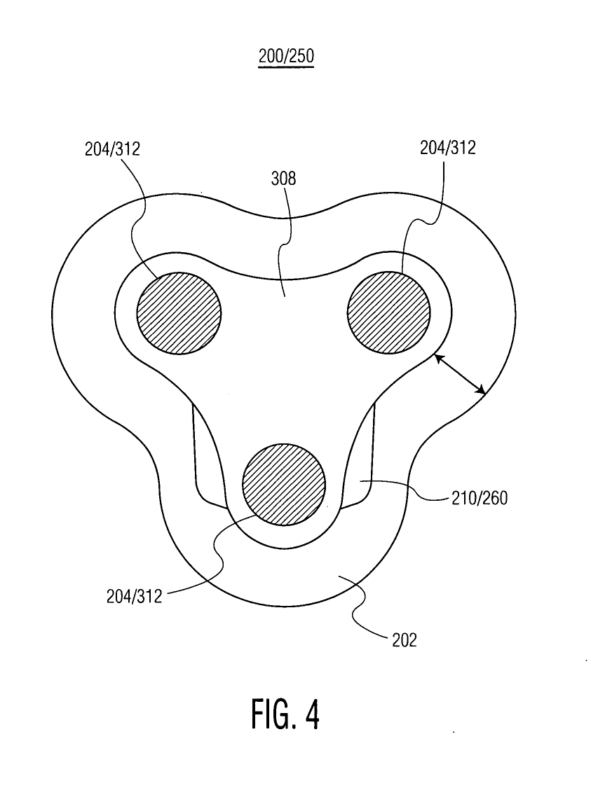

FIG. 4 is a plan view of the medical sensor of FIG. 2.

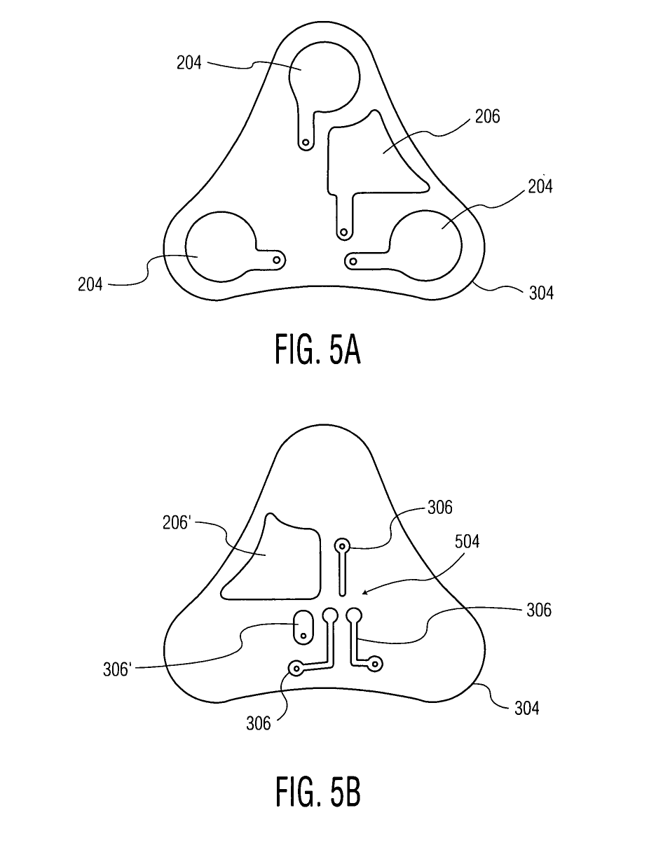

FIGS. 5A and 5B are plan views of a pattern of conductive material according to an embodiment of the present invention for an electrode layer of the medical sensor of FIG. 2.

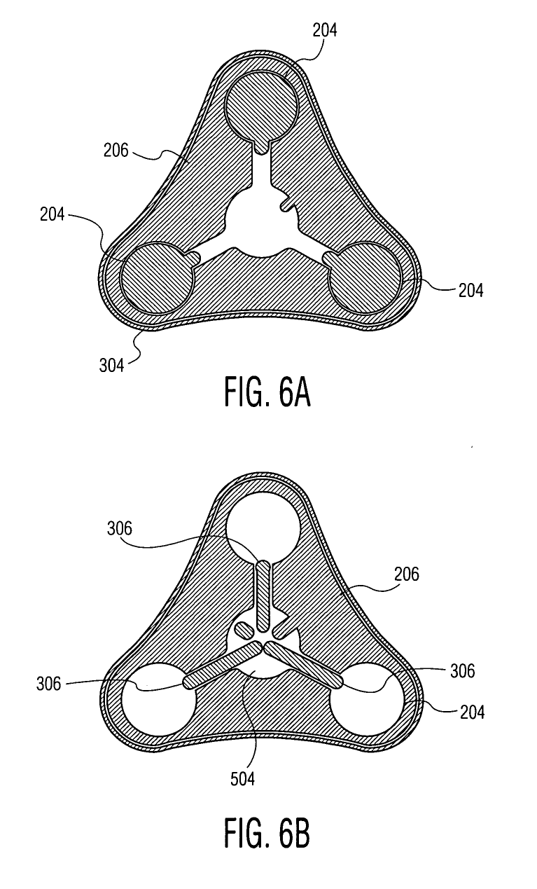

FIGS. 6A and 6B are plan views of a pattern of conductive material according to another embodiment of the present invention for an electrode layer of the medical sensor of FIG. 2.

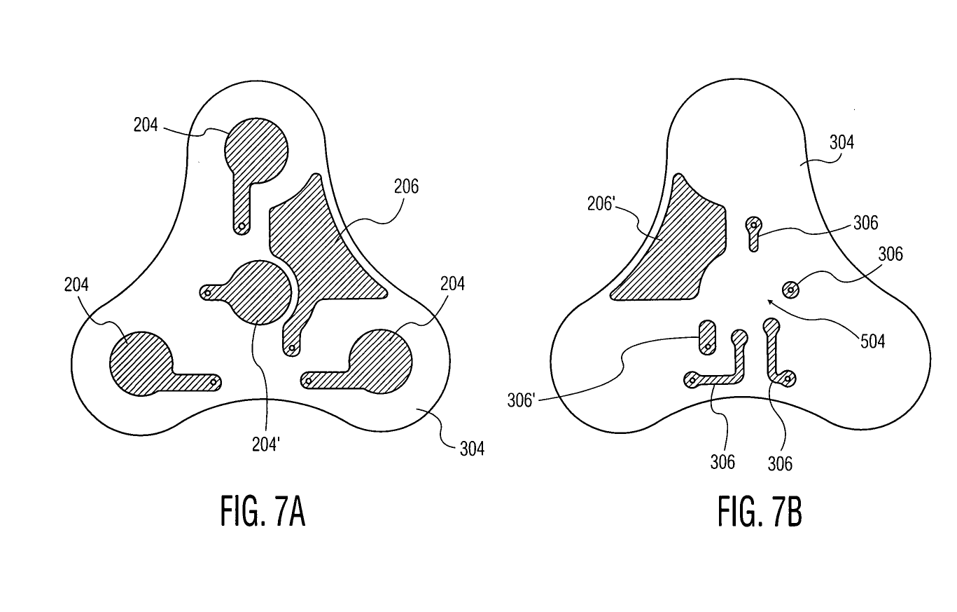

FIGS. 7A and 7B illustrate an electrode layer with four electrodes.

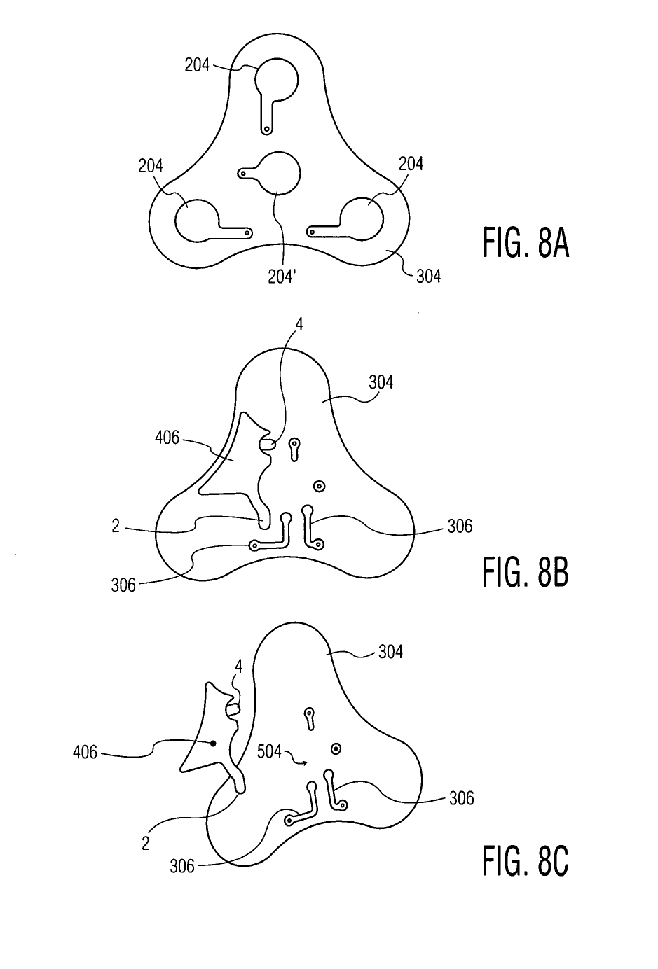

FIGS. 8A, 8B, and 8C illustrate an electrode layer with an integral, separately bonded motion sensor.

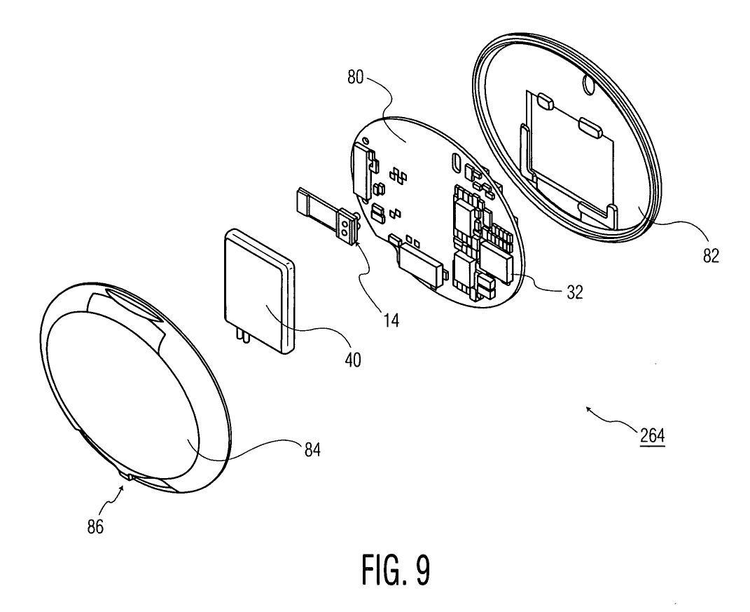

FIG. 9 illustrates a monitor/recorder device for a patient-worn sensor which has a motion sensor integral to the device.

Certain details are set forth below to provide a sufficient understanding of the invention. However, it will be clear to one skilled in the art that the invention may be practiced without these particular details. Moreover, the particular embodiments of the present invention described herein are provided by way of example and should not be used to limit the scope of the invention to these particular embodiments.

FIG. 2A illustrates a medical sensor 200 according to an embodiment of the present invention positioned on a patient 102. As will be described in more detail below, the medical sensor 200 includes a plurality of electrodes 204 for sensing, among other things, the patient's cardiac rhythms as well as a motion sensor 206 that detects patient movement and translates the patient motion into electrical signals that are provided to the monitor/recorder 110. In the embodiment of the medical sensor 200, the motion sensor 206 is integrated in the medical sensor with the electrodes 204. Electrical signals detected and generated by the medical sensor 200 are provided to the monitor/recorder 110 through cable 220 and connector 222. The cable 220 is connected to the medical sensor 200 through connector 210. The medical sensor 200 is adhesively attached to the patient 102 by a flexible retention seal 202. Preferably, the retention seal and adhesive are formed from materials that allow the medical sensor 200 to remain adhered to the patient 102 while in motion and during activity. Such materials are known to those ordinarily skilled in the art, and consequently, in the interest of brevity, a more detailed description of such materials will not be provided herein.

As shown in FIG. 2A, the medical sensor 200 is relatively compact and does not use a plurality of wires for connecting to the monitor/recorder 110, as with the conventional configuration of electrodes shown in FIG. 1. Additionally, the medical sensor 200 includes a motion sensor 206 formed proximate the electrode 204, and is preferably integrated in the medical sensor 200. The information obtained by the motion sensor 206 can be used by the monitor/recorder 110 to gauge patient health. For example, the information can provide an indication if the patient is conscious or unconscious, breathing or not breathing, walking or still. The patient motion data can also be correlated with the ECG waveform to analyze whether to administer cardiopulmonary resuscitation (“CPR”) or defibrillation.

FIG. 2B illustrates a medical sensor 250 according to another embodiment of the present invention positioned on the patient 102. The medical sensor 250 is similar to the medical sensor 200 in that it includes a plurality of electrodes 204 and a motion sensor 206, and is adhesively attached to the patient 102 by a retention seal 202. As with the medical sensor 200, the motion sensor 206 is preferably integrated in the medical sensor 250 with the electrodes 204. In contrast to the medical sensor 200, however, the medical sensor 250 includes a clip 260 that can be used to removably attach a miniature monitor/recorder device 264. The clip 260 is formed with conductive traces that are connected to the miniature monitor/recorder device 264 when it is clipped into place, thereby allowing electrical signals detected and generated by the medical sensor 250 to be provided to the monitor/recorder device 264. As with the medical sensor 200, the medical sensor 250 is relatively compact and does not have a plurality of wires extending across the torso of the patient 102. Additionally, having a miniature monitor/recorder device 264 clipped to the medical sensor 250, provides a compact medical monitor/recorder system 264 that can be readily worn by the patient 102 and avoids the issues associated with conventional monitor/recorder systems and electrode configurations. In another embodiment, the miniature monitor/recorder device 264 includes a motion sensor, alternatively or in addition to the motion sensor 206, that detects patient motion. Although not integrated in the medical sensor 250 with the electrodes 204, the miniature monitor/recording device 264 is firmly attached to the patient 102 by way of the clip 260. Thus, the motion sensor located in the monitor/recording device 264 more accurately detects patient motion than if located in a convention recorder 110 (FIG. 1), which as previously discussed, is worn on the belt 108 or carried on a strap over the shoulder.

FIG. 3 is an exploded isometric diagram of the medical sensors 200 and 250. An electrode layer 304 includes conductive material formed on a dielectric film. The electrodes 204 and conductive traces 306 are formed from the conductive material using conventional processes known in the art. In the embodiment shown in FIG. 3, the motion sensor 206 is formed from regions of conductive material that are formed on opposite sides of the dielectric film resulting in a capacitor structure. Preferably, the conductive film has piezoelectric properties so that movement of a patient wearing the medical sensor 200/250 will be translated into electrical signals. An example of a material that can be used for the conductive material of the layer 304 is polyvinylidene fluoride (“PVDF”), a piezoelectric polymer. PVDF can be used to form flexible and light weight conductive material for the layer 304. The motion sensor can alternatively be made of other piezoelectric materials such as diced or composite PZT ceramic.

A frame 308 is included in the medical sensor 200/250 to provide structural support. The frame 308 is flexible and resilient, allowing the medical sensor 200/250 to bend as the patient moves. An example of a suitable material for the frame 308 is silicone. The frame 308 includes holes 310 which are aligned with the electrodes 204 that are formed on the layer 304. An adhesive material can be applied to the frame 308 on the side opposite of the layer 304 so that when the medical sensor 200/250 is applied to the patient 102 the frame 308 as well as the retention seal 202 are adhesive. Hydrogel 312 is included to provide a conductive coupling medium with the patient when the medical sensor 200/250 is attached. The hydrogel 312 is positioned in the holes 310 and are in contact with the electrodes 204. As a result, when the medical sensor 200/250 is placed on a patient, an electrical connection between the electrodes 204 and the patient are formed.

The layer 304, frame 308, and hydrogel 312 are adhered to the adhesive side of the retention seal 202. A hole 314 in the retention seal 202 allows the conductive traces 306 of the layer 304 to be contacted by the connector 210 for the medical sensor 200 or by the clip 260 for the medical sensor 250. The connector 210/clip 260 is attached to the retention seal 202 using an adhesive, or other process that provides the connector 210/260 to remain electrically coupled to the conductive traces 306 and firmly affixed. A release liner 316 is used to prevent the medical sensor 200/250 from being adhered prior to use and is removed when the medical sensor 200/250 is attached to the patient 102. Although not shown in FIGS. 2A, 2B and 3, the medical sensor 200/250 can also be configured to have a connector, such as a clip connector, that is removably connected so that the medical sensor 200/250 can be first placed on the patient 102 and then connected to the cable 220.

FIG. 4 illustrates the medical sensor 200/250 as viewed from the adhesive side of the retention seal 202 and frame 308 after the release liner 316 has been removed. As shown in FIG. 4, the electrodes 204 are arranged in a triangular configuration. The regions of conductive film that are used for the motion sensor 206 (not shown in FIG. 4) can be generally disposed in the triangular region formed by the arrangement of electrodes 204. By virtue of the piezoelectric properties of the conductive material used in forming the motion sensor 206 and the flexible and resilient nature of the medical sensor 200/250, as the patient 102 moves, likely causing the medical sensor 200/250 to bend and deflect, electrical signals will be generated. As previously discussed, the electrical signals can be used as an indicator of patient health. For example, if motion is sensed, there is a likelihood that the patient is active, and is not in cardiac arrest. Additionally, when related to the patient's cardiac rhythm, the sensed motion can serve as a quality indicator of the monitored and recorded cardiac signals.

FIGS. 5A and 5B illustrate patterns of conductive material formed on a dielectric film for the electrode layer 304 according to an embodiment of the present invention. As previously discussed, an example of the conductive material is PVDF. FIG. 5A illustrates a pattern for a first side of the layer 304 and FIG. 5B illustrates a pattern for a second opposite side of the layer 304. The first side includes conductive regions representing the electrodes 204 and the motion sensor 206. The second side includes a conductive region 206′ (the second capacitive plate) for the motion sensor 206 and conductive regions for the conductive traces 306. The motion sensor 206, as previously discussed, is formed from two or more conductive regions formed in a capacitor arrangement. With this structure, the motion sensor 206 as shown in FIGS. 5A and 5B translates motion (due to stretching, bending and deflection of the conductive regions on the first and second sides) into electrical signals. The conductive traces 306 are configured with printed through-hole vias to provide electrical coupling from the electrodes 204 and the motion sensor region 206 formed on the first side to a generally central region 504 on the second side, from which electrical connections can be made through the hole 314 to the connector 210/clip 260. One of the conductive traces 306′ is formed to provide coupling from the motion sensor region 206 on the first side of layer 304 to the generally central region 504 on the second side. The conductive region 206′ and traces 306, 306′ can be coupled to the connector 210 (FIG. 2A) or to the clip 260 (FIG. 2B), or to another coupling mechanism.

FIGS. 6A and 6B illustrates patterns of conductive material formed on a dielectric film for the electrode layer 304 according to another embodiment of the present invention. FIG. 6A illustrates a pattern for a first side of the layer 304 and FIG. 5B illustrates a pattern for a second opposite side of the layer 304. The first side includes conductive regions representing the electrodes 204 and the motion sensor 206. The second side includes a conductive region for the motion sensor 206 and for the conductive traces 306. The regions of conductive material on the first and second sides for the motion sensor 206 are arranged to provide a capacitor structure. The conductive traces 306 are configured to provide electrical coupling by means of printed or plated through-hole vias from the electrodes 204 and motion sensor 206 formed on the first side to a generally central region 504 on the second side. One of the conductive traces 306 is formed to provide coupling to the motion sensor 206 in the generally central region 504 on the second side.

As with the patterns of FIGS. 5A and 5B, the patterns of FIGS. 6A and 6B provide electrodes 204 that are arranged in a triangular configuration, and the conductive traces 306 provide coupling to the electrodes and the motion sensor 206 to a generally central region. In contrast to the patterns of FIGS. 5A and 5B, however, the patterns of FIGS. 6A and 6B for the regions of conductive material on the first and second sides for the motion sensor 206 generally cover a larger region of the layer 304, namely, a region from the perimeter of the layer 304 to the central region 504. Using the same conductive material for the patterns of FIGS. 5A, 5B and FIGS. 6A, 6B will provide motion sensors 206 having different levels of sensitivity due to the difference in the area of the capacitive regions. Generally, the motion sensor 206 formed using the patterns of FIGS. 6A, 6B is more sensitive than one formed using the patterns of FIGS. 5A, 5B. As illustrated by the two patterns for the motion sensor 206, the level of sensitivity of the motion sensor 206 can be adjusted based on the size of the regions of conductive material on the first and second sides of the layer 304 that are used to form the motion sensor 206. In one embodiment, the sensitivity of the motion sensor is sufficient to detect cardiac pulses of the patient wearing the medical sensor. Although adjusting the sensitivity of the motion sensor 206 by adjusting the size of the regions of conductive material has been described herein, other known techniques can be used as well. The particular technique employed may depend on the type of motion sensor used.

FIGS. 7A and 7B illustrate first and second sides, respectively, of another example of an electrode layer 304 of the present invention. In this example the layer 304 has the motion sensor 206 and three patient electrodes previously discussed. In addition this example has a fourth patient electrode 204′ centrally located on the first side of the layer 304 as shown in FIG. 7A. As can be seen in FIG. 7B, the traces 306, 306′ and motion sensor region 206′ surround the central region 504 of the second side of the electrode layer, from which connections can be made to other electrical conductors or components of the wearable patient monitor.

FIGS. 8A, 8B, and 8C illustrate another example of an electrode layer 304 of the present invention. In this example the layer 304 has four patient electrodes 204 as discussed above. However the motion sensor 406, rather than utilizing the layer 304 material for the capacitive dielectric, is a separate unit with its own dielectric separate from that of layer 304. As shown in FIG. 8C, the separate motion sensor 406 is placed in this example on the second side of the layer 304 and laminated or bonded in place as shown in FIG. 8B. From its location on the second side of the layer 304 connections can be made from the motion sensor extension traces 2,4 to other conductors or components of the patient monitor. FIG. 9 is an exploded view of a monitor/recorder device 264 with an integral motion sensor 14 in accordance with the principles of the present invention. The device 264 has a clamshell case of two halves 82 and 84. On the lower edge of the case half 82 is a connector 86 that connects to a mating connector of the connector 210/clip 260. The electrical components of the device are located on a printed circuit assembly 80, including in this example the piezoelectric motion sensor 14. A battery 40 is located between the printed circuit assembly and the case half 84. The piezoelectric motion sensor 14 may be located on the printed circuit assembly 80 as shown in this illustration, or may be attached to a case half 82 or 84 to take advantage of the acoustic properties of the case and better transmit motion of the patient to the sensor 14.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A medical sensor which attaches to the body of a patient, comprising:
at least one electrode located on a substrate and configured to be attached to a patient for medical monitoring;
a motion sensor located on the substrate and configured to detect patient motion and provide electrical signals in response thereto; and
a processor electrically coupled to the electrode and motion sensor to receive signals therefrom.

2. The medical sensor of claim 1 wherein the motion sensor is integrated onto the common substrate with the electrode.

3. (canceled)

4. The medical sensor of claim 1 wherein the at least one electrode comprises three or more electrodes.

5. The medical sensor of claim 1 wherein the motion sensor comprises a piezoelectric motion sensor configured to translate patient motion into electrical signals.

6. The medical sensor of claim 5 wherein the piezoelectric motion sensor is formed from a polyvinylidene fluoride (PVDF) layer integrated onto the substrate.

7. The medical sensor of claim 6 wherein the electrode comprises a plurality of electrodes attached to the substrate, and wherein the piezoelectric motion sensor comprises a PVDF layer laminated onto the substrate.

8. The medical sensor of claim 7, wherein the piezoelectric motion sensor comprises first and second PVDF layers laminated in opposition to each other on opposite sides of the substrate.

9. (canceled)

10. The medical sensor of claim 1, further comprising:
an adhesive layer configured to adhere the medical sensor to the patient.

11. (canceled)

12. A medical sensor, comprising:
a plurality of electrodes configured for attachment to a patient and operable to electrically couple electrical signals to and from the patient; and
an integral motion sensor configured to sense patient motion and provide signals in response thereto,
wherein the integral motion sensor and the plurality of electrodes are located on a common substrate of the medical sensor.

13. (canceled)

14. (canceled)

15. The medical sensor of claim 12 wherein the integral motion sensor comprises a piezoelectric motion sensor configured to translate patient motion into electrical signals.

16. (canceled)

17. The medical sensor of claim 13, further comprising:
an adhesive layer configured to adhere the medical sensor to the patient.

18-22. (canceled)
